(19) United States
(12) Patent Application Publication
(10) Pub. No.: US 2013/0028987 A1
(43) Pub. Date: Jan. 31, 2013

(54) Method of treating psoriasis

(71) Applicant: Madalene C.Y. Heng, Northridge, CA (US)

(72) Inventor: Madalene C.Y. Heng, Northridge, CA (US)

(21) Appl. No.: 13/136,261

(22) Filed: Jul. 25, 2011

(51) Int. Cl. A61K 33/30; A61K 33/20; A61P 31/04; A61P 3/02; A61P 17/06; A61P 31/10

(52) U.S. Cl. 424/643; 424/661

(57) ABSTRACT

A method treating psoriasis and more specifically toward a protocol that involves the use of a specialized curcumin gel composition, the use of topical steroid cream, the avoidance of contact with allergens, and lactose-free diet and treatment of bacterial superinfection with antibiotics and bleach/chlorox baths.

### BACKGROUND OF THE INVENTION

1. Field of the Invention

The present invention relates to methods of treating psoriasis and more specifically toward a protocol that involves the use of a specialized curcumin gel composition, the use of topical corticosteroid preparation (cream, ointment, solution or foam/spray), the avoidance of contact with topical allergens, lactose-free diet and treatment of bacterial superinfection with antibiotics and bleach/chlorox baths.

2. Description of the Prior Art

Psoriasis is an inflammatory skin disease of unknown cause which is usually chronic, frequently recurrent and acute in nature. This skin disease produces lesions that occur predominantly at certain sites, such as elbows, knees and scalp, although other areas of the body may be also inflamed. Psoriasis consists of dull, red, well-defined patches that are usually covered by distinctive silvery scales which, when removed, disclose tiny capillary bleeding points. These lesions spread by peripheral extension and may involve huge areas of the body. The patches are not constant in size, shape and location. It is believed that the psoriasis is activated by external stimuli, such as trauma or contact allergens, as well as by certain bacteria, which trigger the psoriatic process in genetically predisposed individuals.

There are a number of different treatment options for psoriasis. Typically topical agents are used for mild disease, phototherapy for moderate disease, and systemic agents for severe disease. U.S. Pat. No. 7,556,818 to Heng (the instant inventor) provides a curcumin gel treatment for psoriasis, the composition of which is fully incorporated by reference and used in the within protocol.

### SUMMARY OF THE INVENTION

The preferred embodiment is a method of treating psoriasis comprising the steps of: eliminating lactose-containing foods from the diet of the patient; providing oral antibiotics for the treatment of any present causative infection; providing oral anti-yeast medicine; providing topical ketoconazole (creams, shampoos) for topical use; providing clobetasol (in a gel, solution, cream, ointment, foam or spray) for topical use; and providing curcumin gel in an alcohol solution for topical use. Bleach baths are also used in order to penetrate the holes within the psoriatic scales which are colonized by bacteria.

The above embodiment can be further modified by defining that oral calcium supplements are provided.

The above embodiment can be further modified by defining that oral vitamin D supplements are provided.

The above embodiment can be further modified by defining that oral zinc supplements are provided.

The above embodiment can be further modified by defining that oral zinc supplements are provided at 40-60 mg daily.

The above embodiment can be further modified by defining that the oral antibiotic is taken from the group including: cephalexin, clindamycin, trimethoprim/sulfamethoxazole, or ciprofloxacin or levofloxacin in order to kill Staphylococcus aureus and or Streptococcus sp.

The above embodiment can be further modified by defining that the antibiotic may be intravenously administered such as vancomycin to kill MRSA (methicillin resistant Staphylococcus aureus).

The above embodiment can be further modified by defining that an antifungal medication can be administered orally in the form of fluconazole DIFLUCAN® or topically in the form of topical ketoconazole cream for the trunk, face, arms and legs.

The above embodiment can be modified by further defining that the ketoconazole can be used as a shampoo for the scalp.

### BRIEF DESCRIPTION OF THE DRAWINGS

FIG. 1 is a diagram that summarizes the defective switch off mechanism in psoriasis according to the findings of the instant inventor.

### DETAILED DESCRIPTION OF A PREFERRED EMBODIMENT

This embodiment is based on concepts and theory by the instant inventor that the psoriatic gene fails to switch-off the elevated phosphorylase kinase induced by injury, such as trauma, allergic reactions and infections. The genetic defects can be due to abnormalities in genes mapped to the distal end of the 17th chromosome (i.e. 17q), or to abnormalities in genes mapped to the distal end of the 16th chromosome (i.e. 16q). The 17q gene anomaly encodes an abnormal cyclic AMP (Type II) which fails to switch-off the activity of phosphorylase kinase. The 16q genetic anomaly encodes for the beta subunit of phosphorylase kinase, which contains the receptor for the Type II cAMP protein kinase. Both genetic abnormalities, i.e. anomalies either in the ligand (Type II cAMP protein kinase, encoded by 17q) or its receptor (located on the β subunit of phosphorylase kinase encoded by 16q), will result in failure to switch-off the elevated phosphorylase kinase activity induced by injury/allergic reactions and infection.

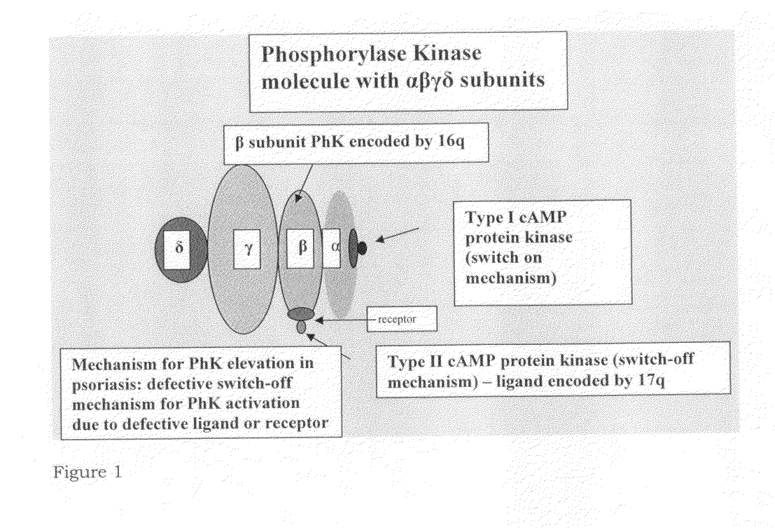

FIG. 1 summarizes the defective switch off mechanism in psoriasis according to the findings of the instant inventor. The treatment of psoriasis according to the protocol is based on an understanding of this concept. Curcumin, a selective and non-competitive inhibitor of phosphorylase kinase opposes phosphorylase kinase activity and helps switch off the persistently elevated phosphorylase kinase activity in psoriatic skin, thus resulting in resolution of psoriasis.

After injury, such as trauma, allergic reactions and/or infections, phosphorylase kinase activity is induced. Elevated phosphorylase kinase activity not only breaks down glycogen into ATP needed for multiple reactions, but activates IkBa kinase, a key enzyme responsible for activating NF-kB, a transcription activator. Activation of NF-kB then goes on to turn on over 200 genes responsible for cell proliferation, cell cycling, inhibition of apoptosis etc., resulting in the formation of PCNA+ (proliferating cell nuclear antigen), which is detected by the immunocytochemical marker, Ki-67+. The Ki-67+ cells are capable of producing new cells rapidly and their increased numbers in the basal keratinocyte layers of the psoriatic epidermis are responsible for the hyperproliferation characteristic of the psoriatic disease.

Phosphorylase kinase is activated by “injury stimuli”, which releases Type I cAMP protein kinase, which further activates the phosphorylase kinase molecule by a conformational change in the molecule, which allows access to phosphorylation sites for reactivity. After the “wound” is healed, the activation of Type II cAMP protein kinase “closes up” the molecule, thus switching off phosphorylase kinase activity by obscuring these phosphorylation sites. The abnormal Type II protein kinase in psoriatic individuals results in an inability to switch off phosphorylase kinase activity. A second possibility lies in abnormality in the beta subunit of phosphorylase kinase, which contains the receptor for binding to the Type II cAMP protein kinase ligand With either abnormality, there is defective ability to deactivate the elevated phosphorylase kinase activity induced following injury. Without the capacity to switch-off proliferative activity, the psoriatic skin is, therefore, chronically in the proliferating mode. Curcumin gel is a selective phosphorylase kinase inhibitor that switches off phosphorylase kinase activity, thus allowing the Ki-67+ psoriatic cells to undergo apoptosis, leading to normalization of its proliferative activity, and resolution of the psoriatic epidermis.

Although the psoriatic gene is present at birth, a person does not develop psoriasis until precipitated by trauma, such as injury, allergic reactions and/or infections.

The location of psoriasis gives a clue as to what precipitates or aggravates the disease. When it appears on the scalp, the precipitating factor can be overgrowth of Pityosporum ovale, a lipophilic yeast which feeds on the oils secreted by sebaceous glands. The population of P. ovale is increased by increased oil production, which is aggravated by stress, and by lactose intolerance. When psoriasis appears on the scalp, another precipitating factor could be contact allergy to black or brown hair dyes (paraphenylene diamine). Psoriasis can also be precipitated on the scalp and elsewhere by nickel allergy through exposure to nickel filings brought to the scalp by touching with nickel (coins and keys)-contaminated fingers. Scalp psoriasis can be aggravated by the presence of Staphylococcus aureus colonizing the porous skin scales. When it appears on the scalp, it may also be that sufferers have lesions on the face near the hair-line, eyebrows, paranasal cheeks. Also, it may show up near the ears as well as behind the ears.

When the psoriasis appears on the trunk and limbs, the affected areas are usually in areas in contact with elastic in clothing, such as bra areas, around waist areas, areas in contact with socks and/or stockings, and in areas in contact with leather products such as upholstery in cars and chairs, as well as skin in contact with black or brown dyes in clothing, or when the black dye leaches out of the hair during showers.

The common allergens and aggravating factors include elastic antioxidants in latex, spandex underwear, clothing and socks, paraphenylene dyes in black/brown clothing and hair dyes, nickel from coins and keys, leather products, and Staphylococcus aureus/MRSA bacterial superinfection.

When appearing on hands and fingers, especially around the nails, exposure is usually from nickel from coins and/or keys contacted by putting hands in pockets containing coins and keys. In the web spaces and dorsum of hands and fingers, precipitating factors include elastic antioxidants from latex gloves, nickel filings, detergents, trivalent chromates found in gasoline, leather products, printer's ink, motor oils, brake fluid, lubricating oils, and cement. When appearing on the palms, precipitating factors include paraphenylene diamine (black) dyes, and trivalent chromates from leather products in black leather steering wheel covers, black/leather handles of tennis rackets, black/leather handles of golf clubs. The patient may also be allergic to paints, varnishes and/or glues which may precipitate psoriasis on the hands.

When appearing on the dorsum of the feet, the precipitating factors include exposure to trivalent chromates in leather shoes and other foot-wear or paraphenylene dyes, and/or elastic material in shoes. On the soles, the exposure can be to leather insoles and/or neoprene glues that glue insoles to sock linings. In cases like this, treatment can take the form of using plastic wrap to insert between the footwear and the skin of foot to prevent the allergen from contacting the skin.

In flexural areas, such as the axilla, groin and/or perianal areas, aggravating factors include Candida albicans, obesity or diabetes. It may also be aggravated by lactose intolerance, which results in zinc deficiency. Since human mother's milk contains galactose and cow's milk contains lactose, humans cannot secrete enough lactases in the small intestine to digest the lactose we ingest. As a result, the undigested lactose spills into the colon, and, through increasing the osmotic pressure in the colon, interferes with the colonic bacteria. The interaction of the colonic bacteria with undigested lactose in the colon results in dead colonic bacteria, which release lipopolysaccharides (LPS) from the bacterial membranes. LPS is a potent superantigen, which activates large numbers of T lymphocytes, and stimulates the formation of high levels of TNF-alpha, which causes worsening of psoriasis.

Treatment for psoriasis should include the substitution of soy products for lactose-containing foods, and the dietary supplementation of calcium and vitamin D. Goat cheese may also be substituted, which does contain lactose, but is more digestible.

The presence of psoriasis can also be linked to the presence of bacterial superinfection, i.e., Staphylococcus aureus in plaque psoriasis, especially if the lesions are red and itchy. This is due to superantigens (Protein A, enterotoxin A, B and C) released from the cell wall of Staphylococcus aureus by the presence of proteases in serum, causing erythroderma and dissemination of the psoriatic lesions just like in poison oak dermatitis.

The Staphylococcus aureus bacteria lives inside the holes in the porous psoriatic scales just like bacteria inside the pumice stone. This is the reason why it is recommended to give some form of oral antibiotics to every psoriatic patient. However, the bacteria within the porous psoriatic scales are not killed by the oral REFLEX® alone, since the scales are essentially outside the body. They also need a topical treatment e.g. bleach/chlorox baths. The chlorine water soaks into the scales, and kills the bacteria within.

In a bleach/chlorox bath, the chlorine concentration resembles that within a typical swimming pool, and should not be toxic, even for babies. One eighth of a cup of chlorox bleach is added to a half-filled full bath tub of tepid water. The water must not be too warm or the chlorine will irritate. The chlorox is mixed in with the fingers. Then the patient gets into the bath tub and soaks for 5 minutes, pouring the chlorine water over the head and trunk that is not under water. This is done because Staphylococcus aureus can be cultured from normal skin (in 14% in one study). The patient should keep his head down (i.e. chin to chest), so as not to get the water into his ears. While in the bath tub, there should be no soap as the formation of soap bubbles interfere with the ability of chlorine to penetrate into the extremely tiny pores inn the psoriatic scales. Since Staphylococcus aureus has been cultured from the nares in 50% of patients, the nose should be rinsed with chlorine water as well.

After the bath, the patient showers off the chlorine with soap and water, and shampoos his hair. He uses a fresh towel daily, changes into fresh clothes and socks daily, and changes his bed sheets and pillow cases daily as these are full of bacteria.

For oral treatment, REFLEX® (cephalexin) or other appropriate antibiotic directed against the bacterial superinfection is used, until the psoriasis is completely clear (usually in 4 months, longer for palms and soles). If skin cultures are positive for MRSA infection (methicillin resistant Staphylococcus aureus infection), other appropriate antibiotics, or even intravenous vancomycin may be necessary. For yeast infections, antifungal agents, such as Diflucan® at 200 mg once a week for scalp and groin may be prescribed until the psoriasis is clear/completely resolved (usually 4 months for the scalp, about 2 or more months for groin and axilla). Zinc supplements are also useful since psoriasis uses up zinc stores. At least 40 mg daily of zinc may be helpful.

For topical treatment, for the face and flexural areas, apply ketoconazole cream 2% mixed with triamcinolone cream 0.1% or other mild steroid preparation once or twice a day and curcumin gel after a bath at night. On the scalp, apply clobetasol solution 0.05%/Clobex® spray in the mornings between the hair roots and ketoconazole shampoo 2% at night and curcumin gel after drying hair in the evenings. For the trunk, limbs, hands and feet, use ketoconazole cream 2% mixed with clobetasol cream 0.05% morning and noon, and curcumin gel after a bath at night.

Curcumin gel is used every night after the bath or shower. Described here is a unique method of getting the curcumin gel to penetrate the psoriatic scales. Because the psoriatic scales are full of air-filled holes, these air-filled holes prevent the curcumin gel from penetrating through and under the psoriatic scales, which resemble pumice stone in that they are full of holes. Pumice stone used to be molten liquid rock inside the volcanic crater, with air bubbles boiling through the hot liquid. After the volcanic eruption, the pumice cooled and enshrined the air bubbles inside. The air bubbles reflect light and pumice is white in color just like the psoriatic scales. If you rub curcumin gel onto the pumice stone, the curcumin gel will just stick to the outside of the stone. However, if you pour liquid onto the pumice stone, the liquid seeps in through the holes, and displaces the air bubbles. If you soak the psoriatic scales with rubbing alcohol so that the scales no longer have the white appearance, and then massage the curcumin gel into the wet alcohol, the wet alcohol will drag the curcumin gel through and under the psoriatic scales. Curcumin gel is only soluble in alcohol, and you can only use any alcohol, including rubbing alcohol to displace the air bubbles in the scales. The two elements for success is the pretreatment with alcohol, and then massaging the curcumin gel into the wet alcohol. It is important not to allow the alcohol to dry out before massaging in the curcumin gel, or the air bubbles will reform and prevent the penetration of the curcumin gel through the scales. A swab is used to transfer the curcumin gel onto the skin. It is important not to re-use the swab after the initial use so as not to infect the contents of the jar. It is likewise important to remember to close the lid of the jar tightly after use.

Patients usually are 65-70% improved in 4 weeks, 85% improved in 8 weeks, 95% in 3 months and almost totally clear in 4 months. The palms and soles take longer because they have 200 layers of stratum corneum instead of 10 layers of stratum corneum to replace. It takes 60 days to replace the first layer of stratum corneum.

Corticosteroids (both oral and topical) do not suppress phosphorylase kinase, and do not kill off the Ki-67+ epidermal cells that cause the skin to grow rapidly. The disease recurs when treatment is stopped. If the protocol described herein is followed correctly and the treatment continues until the Ki-67+ cells are normalized, the disease does not recur after the treatment is tapered off. This is, of course, with the proviso that the injury, precipitating factors and/or aggravating factors are removed and/or treated.

If spots appear about the size of a dime or nickel, without large spots, it is the guttate type of psoriasis. This is usually precipitated by streptococcal infection, although other causes are possible, e.g. herpes simplex infection, hepatitis and/or penicillin allergy.

To get a streptococcal infection the gene HLA-B16 must be present. The psoriasis itself is due to another gene (mapped to the distal end of the 17th chromosome). Streptococcal infection may originate from the scalp, the throat, ears or sinuses. The scalp may also be infected by Staphylococcus aureus, by a yeast called Pityosporum ovale, and aggravated by nickel from coins and keys by touching with your fingers.

If there are lesions present in the groin, axilla and/or between the buttocks, there may be lactose intolerance. In fact, the whole human race is lactose intolerant in varying degrees, since cow's milk is intended for baby cows rather than humans. However, some races or individuals are more lactose intolerant than others. The lactose in the colon causes stress to the bacteria in the colon that are trying to digest cellulose since human beings are incapable of digesting cellulose. The inflammation in the bowel causes colitis, which causes zinc deficiency, which causes proneness to yeast and bacterial infection. In the scalp and face, the yeast is Pityosporum ovale; elsewhere (groin, axilla, between the buttocks, between the fingers and toes etc) the yeast is Candida albicans.

The protocol and treatment of the instant invention involves the following steps. First, go on a lactose free diet, eliminating cheese, ice cream, yogurt, sour cream, cream soups containing milk, pastries containing milk, sauces containing milk, etc. and substitute soy products and/or goat cheese instead. Calcium and vitamin D supplements are recommended, as well as zinc supplements (40-60 mg daily).

Second, use oral antibiotics to treat any streptococcal infection and/or Staphylococcal or MRSA infection, such as oral cephalexin/KEFLEX® 500 mg, 2 capsules twice daily until clear, typically after 4 months. The bacteria in the scales are killed by bleach/chlorox baths.

Third, orally treat yeast with oral fluconazole/DIFLUCAN® 200 mg tabs—one tablet per week until clear, typically after 4 months.

Fourth, use a ketoconazole shampoo 2% for the yeast in the scalp.

Fifth, on the scalp, use a clobetasol solution 0.05% or clobetasol spray 0.05% in the mornings, massaging between the hair roots. Then use curcumin gel at night after a shower, and after drying the hair. Wet the scales with a little rubbing alcohol, and while the scales are still wet, massage curcumin gel into the wet alcohol in order to get the curcumin gel to penetrate under the scales. This may also be done for the trunk and limbs as well at night after the shower.

Sixth, for the trunk and limbs, use a combination of clobetasol cream 0.05% mixed with ketoconazole cream 2% on the palm of the hand, and then apply to the patches on the trunk and limbs. Do this morning and noon if possible.

Seventh, after a bleach/chlorox bath and shower at night, apply curcumin gel, massaging it into the wet alcohol to get the gel to penetrate the psoriatic scales.

The illustrations and examples provided herein are for explanatory purposes and are not intended to limit the scope of the appended claims. This disclosure is to be considered an exemplification of the principles of the invention and is not intended to limit the spirit and scope of the invention and/or claims of the embodiment illustrated. Those skilled in the art will make modifications to the invention for particular applications of the invention.

What is claimed is:

1. A method of treating psoriasis comprising the steps of:
eliminating lactose containing foods from the diet of the patient;
avoiding precipitating factors such as contact allergens,
providing oral/intravenous antibiotics for the systemic treatment of any present causative infection;
providing bleach/chlorox baths to kill the bacteria living inside the psoriatic scales;
providing oral anti-yeast medicine;
providing ketoconazole for topical use;
providing clobetasol or other corticosteroid preparation (creams, ointments, solutions, gels, foam, spray or other preparation for topical delivery) for topical use; and
providing curcumin gel in an alcohol solution for topical use.

2. The method as defined in claim 1 wherein oral calcium supplements are provided.

3. The method as defined in claim 1 wherein oral vitamin D supplements are provided.

4. The method as defined in claim 1 wherein oral zinc supplements are provided.

5. The method as defined in claim 4 wherein oral zinc supplements are provided at 40-60 mg daily.

6. The method as defined in claim 1 wherein said oral antibiotic is taken from the group including KEFLEX®/cephalexin or other appropriate antibiotics aimed at treating Staphylococcus aureus and/or streptococcus sp, and/or MRSA e.g. sulfamethoxazole/trimethoprim, clindamycin, ciprofloxacin, levofloxacin, vancomycin.

7. The method as defined in claim 1 wherein said ketoconazole is provided as a shampoo for the scalp.

8. The method as defined in claim 1 wherein said ketoconazole is provided as a cream for the trunk and limbs.

9. The method as defined in claim 8 wherein other anti-fungal creams, including clotrimazole cream, miconazole cream or other anti-fungal creams are provided.
